(19) United States
(12) Patent Application Publication
(10) Pub. No.: US 2015/0362507 A1
(43) Pub. Date: Dec. 17, 2015

(54) System and Methods of Detecting and Demonstrating Ultraviolet Damage to Hair Via Evaluation of Protein Fragments

(71) Applicant: The Procter & Gamble Company, Cincinnati, OH (US)

(72) Inventors: Michael Glenn DAVIS, Liberty Township, OH (US); Michael Joseph FLAGLER, Cincinnati, OH (US); Jennifer Mary MARSH, Mason, OH (US); Yiping SUN, Mason, OH (US); Tanuja CHAUDHARY, West Chester, OH (US)

(21) Appl. No.: 14/729,666

(22) Filed: Jun. 3, 2015

Related U.S. Application Data
(60) Provisional application No. 62/011,272, filed on Jun. 12, 2014.

(51) Int. Cl. G01N 33/68

(52) U.S. Cl. CPC G01N 33/6851; G01N 2333/4727; G01N 2440/10; G01N 2560/00; G01N 2570/00; G01N 2800/40; G01N 2500/20

(57) ABSTRACT

The present invention is directed to a method to measure ultraviolet or copper damage of hair comprising: eluting a protein fragment from a hair sample with an aqueous solution; extracting the proteins using a solvent; analyzing the protein fragment samples with MALDI-MS; resulting in protein fragment results; identifying presence of a marker protein fragment and identifying what the fragment is by indentifying the amino acid sequence using high resolution Orbitrap-MS wherein the protein fragment is a protein fragment of S100A3.

### FIELD OF THE INVENTION

Embodiments of the present disclosure are directed to a process for measuring the ultraviolet damage to the hair by the evaluation and identification of extracted protein fragments.

### BACKGROUND OF THE INVENTION

Hair damage through protein loss is a known problem; however, most people have no recognition of the amount of protein loss experienced by their hair, or their level of hair health in general. Protein loss may be caused by everyday occurrences and environmental factors such as ultraviolet (UV) ray exposure, bleaching, coloring, perming, straightening, mechanical manipulation, and salt water contact.

Proper hair architecture at the molecular level is an important characteristic of hair that has a healthy look, shine and feel. The hair comprises mostly protein and is not regenerative after it exits the scalp. Therefore, it is valuable to have products which protect the overall protein integrity of the hair. Thus, protection of the hair shaft on the protein and fiber level is important to ensure hair has a healthy look.

Identifying the protein fragments extracted from the hair and correlating the type of protein fragment with a type of hair damage 1) enables a correct identification of the type of damage to the hair, and 2) may provide the information necessary to design products which either prevent the damage, or in the case of bleaches and/or other composition do not generate the damage. Additionally, it is also valuable to identify particular types of hair disease. Hair of individuals with hair diseases, do not react to damage and/or treatments in the same way as normal hair. Therefore, it may be possible to indicate what type of hair disease is present based upon the response of the hair at a protein level to a particular type of damage.

### Weather-Damage Effects

It is widely known that the weathering effects due to environmental factors (exposure to sunlight, air pollutants, wind, sea water, and chlorine in pool water) damage the hair and that such effects can be detected at the morphological level. Further, sunlight, pool water, and cosmetic products such as permanent waves, bleaches, straighteners, and some hair dyes chemically alter hair and increase its propensity to further chemical and mechanical breakdown as evidenced by an increased sensitivity to cuticle abrasion/erosion and fiber splitting. Hair damage from UV-A and UV-B rays is a significant problem for consumers and affects both the physical and cosmetic properties of the hair.

### UV-Damage Effects

It is well established that light radiation (as from the sun) damages hair, degrading the hair proteins, the cell membrane complex lipids and the hair pigments. Both visible light and UV-A/UV-B light damage the hair structure. This damage weakens the cell membrane complex and multiple step fractures can be observed in hair exposed to light radiation.

Further, sunlight and ultraviolet light have been shown to decrease the wet tensile properties of human hair. They relate these effects to the total radiation that the hair is exposed to rather than to any specific wavelength. However, more recently, hair protein degradation by light radiation has been shown to occur primarily in the wavelength region of 254 to 400 nm.

Also, as the protein fragment is identified, products which utilize the available bonds that result from the protein loss, in particular products specialized for specific damage types, can be produced.

### SUMMARY OF THE INVENTION

The present disclosure relates generally to systems and methods for detecting ultraviolet (UV) hair damage by correlating protein fragments extracted from the hair to a type of hair damage.

An embodiment of the present invention is directed to a method to measure ultraviolet damage of hair comprising eluting a protein fragment from a hair sample with an aqueous solution; extracting the proteins using a solvent; analyzing the protein fragment samples with MALDI-MS; resulting in protein fragment results; identifying presence of a marker protein fragment and identifying what the fragment is by identifying the amino acid sequence using high resolution Orbitrap-MS wherein the protein fragment is a protein fragment of S100A3.

A further embodiment of the present invention is directed to a method to identify a treatment for ultraviolet damage of hair comprising applying a treatment composition to hair sample A; apply no treatment composition to sample B; apply ultraviolet light to a hair sample A and hair sample B; eluting a protein fragment from hair samples with an aqueous solution; extracting the proteins using a solvent; identifying or measuring the marker protein fragments by identifying the unique modification patterns which exist in sample wherein the MALDI-MS protein fragment results in a protein fragment of S100A3 wherein Sample A has a decreased intensity in protein fragment of S100A3 compared to Sample B.

### BRIEF DESCRIPTION OF THE DRAWINGS

FIG. 1. This figure is the MALDI mass spectrum of marker protein fragments for Virgin Hair.

FIG. 2. This figure is the MALDI mass spectrum of marker protein fragments for UV treated hair.

FIG. 3. This figure is the MALDI mass spectrum of marker protein fragments for bleached hair.

FIG. 4. This figure is a graph of the measure of the m/z 1278 peak intensity as measured over 0, 25, 50 and 75 hours of UV exposure.

FIG. 5. This figure is a graph of the measurement of the m/z 1278 peak intensity as measured over 0, 20 and 40 hours of UV exposure in hair that has been exposed to increasing copper levels.

FIG. 6. This figure is the MS/MS fragment ion spectrum of the m/z 1278 protein fragment obtained on a high resolution Orbitrap mass spectrometer, showing its sequence as a fragment of S100A3 (Acetylated-ARPLEQAVAAIV-Amide).

FIG. 7. This figure is a graph of the measurement of total protein loss for a shampoo with no chelant (no EDDS) and a shampoo with 0.1% chelant (EDDS).

FIG. 8. This figure is a graph of the measurement of m/z 1278 intensity for a shampoo with no chelant (no EDDS) and a shampoo with 0.1% chelant (EDDS).

### DETAILED DESCRIPTION OF THE INVENTION

As used herein, “hair” means keratinous fibers of the human or animal origin, such as hairs on the head or eyelashes. Furthermore, as used herein, the term “keratinous protein” is understood to mean those proteins present in hair. As used herein, the term “protein fragments” means the amino acids and larger proteins that are damaged and broken off the keratinous protein structure and held within the hair structure by electrostatic interactions, weak hydrogen bonding matrix proteins and lipids, or any other force that does not include incorporation in the keratinous protein structure.

As used herein “marker protein fragment” means the protein fragment which has been correlated to a particular type of hair damage and/or damaging treatment.

As used herein, “elutes,” “eluting,” and the like means removing proteins from hair via contacting hair with an aqueous solution without the addition of any reduction or extraction agents, thereby yielding no modification of the keratinous protein structure and no breaking or reduction of chemical bonds present in the hair sample other than electrostatic interactions, weak hydrogen bonding matrix proteins and lipids, or any other force that does not include incorporation in the keratinous protein structure.

As used herein, “elutable” means protein fragments present in the hair sample that may be removed from the hair structure in an aqueous solution without the addition of any reduction or extraction agents. Furthermore, “elutable” means proteins that may be carried out of the hair structure in an aqueous solution consisting essentially of water without the breaking or reduction of chemical bonds present in the keratinous protein structure other than electrostatic interactions, weak hydrogen bonding matrix proteins and lipids, or any other force that does not include incorporation in the keratinous protein structure.

A method has been developed for detecting and demonstrating hair damage by utilizing an aqueous solution to extract protein fragments from the hair without modifying the keratinous protein structure. Once the protein fragments are extracted from the hair, the protein fragments are analyzed. From the analysis of the protein fragments it is possible to identify the type of damage that has been done to the hair, in particular it is possible to determine the source of the damage to the hair. One such specific marker protein fragment includes those marker protein fragments generated when the hair is exposed to UV light and/or copper. A hair sample can be tested, the protein fragments extracted, and the resulting protein fragments tested using an antibody based detection, and/or a mass spectrometry technique. In one embodiment the protein fragments are evaluated using the Matrix Assisted Laser Desorption Ionization (“MALDI”), also known as the MALDI-TOF Mass Spectrometry “MALDI-MS”. This technique is a soft ionization technique used in mass spectrometry. MALDI-MS can be used for the analysis of biomolecules such as peptides and proteins and large organic molecules such as polymers. In MALDI, the analyte is first co-crystallized with a UV absorbing matrix such as alpha-cyano-4-hydroxycinnamic acid (CHCA), then subjected to pulse laser (YAG or nitrogen laser) radiation. This causes the vaporization/desorption of the analyte/matrix crystals and produces ions which are transmitted into a mass analyzer for detection. In MALDI-TOF, a time-of-flight mass analyzer is used. MALDI-TOF data can be acquired in MS mode to generate molecular weight information (e.g., a peptide) and in MS/MS mode (e.g., a peptide sequence/structure information). Typical MALDI mass spectrum acquisition takes less than a minute so it can be used for fast screening of molecular species in samples of interest. Changes and molecular markers can be detected by comparing the mass spectra acquired in samples treated under different conditions such as virgin hair vs. UV or copper exposed hair.

MALDI-MS can be performed either with or without enzymatic digestion of proteins. The protein fragment test results are then compared to a library of known marker protein fragments to identify what type of hair damage, and in some situations, what is the original source of damage to the hair i.e. bleach. This enables a “fingerprinting” of damage; meaning that if a hair sample is tested and the results include certain marker protein fragments, then the hair sample has been damaged by a particular source.

Additional methods for evaluating the protein fragments include, but are not limited to, liquid chromatography-electrospray mass spectrometry, multiple reaction monitoring (MRM) mass spectrometry, antibodies against the protein fragments could be generated and an ELISA assay could be developed.

Further an iTRAQ (Isobaric Tags for Relative and Absolute Quantitation) method, reagents available through AB-Sciex, Framingham, Mass. can be used to establish covalent amine linkage of an isobaric tag to each lysine side chain and free N-terminal group of a protein fragment. This allows for multiple samples to be run simultaneously through the MALDI-MS. Running multiple samples through the MALDI-MS simultaneously minimizes variations in the test data due to test variability.

Furthermore MALDI-Imaging mass spectrometry can be used to map chemical species directly on surfaces like hair. Peptide fragments on damaged hair can be directly detected and visualized on hair fibers. The marker peptide intensity in a MALDI image that is acquired in the same data acquisition can be compared. For example, single hair fibers from virgin, bleach or UV exposure can be mounted directly on a MALDI plate using a piece of double-sided, conductive adhesive tape. After applying a MALDI matrix like α-cyano-4-hydroxycinnamic acid (CHCA), the hair fiber surface can be analyzed in the imaging mode by rastering the laser beam across the area. Data acquired in the MALDI-Imaging mode can be processed using an imaging software such as Biomap. The ion intensity map on a specific damage marker present on hair fiber surface (e.g., m/z 1278 from UV irradiation and m/z 1037 from bleach) can be generated for visual comparison.

A library of these marker protein fragments can be generated by damaging swatches of hair with a variety of different compositions or treatments and then analyzing the resulting protein fragments in comparison with a similar swatch of hair which has not been damaged. Marker protein fragments can be identified by the MALDI-MS, as it is believed the same marker protein results will be found based upon the type of damage that the hair has experienced. This means that the marker protein fragment is indicative of a type or source of hair damage. Hair damage by UV and/or copper results in particular marker protein fragments, hair damaged by bleach results in particular marker protein fragments etc.

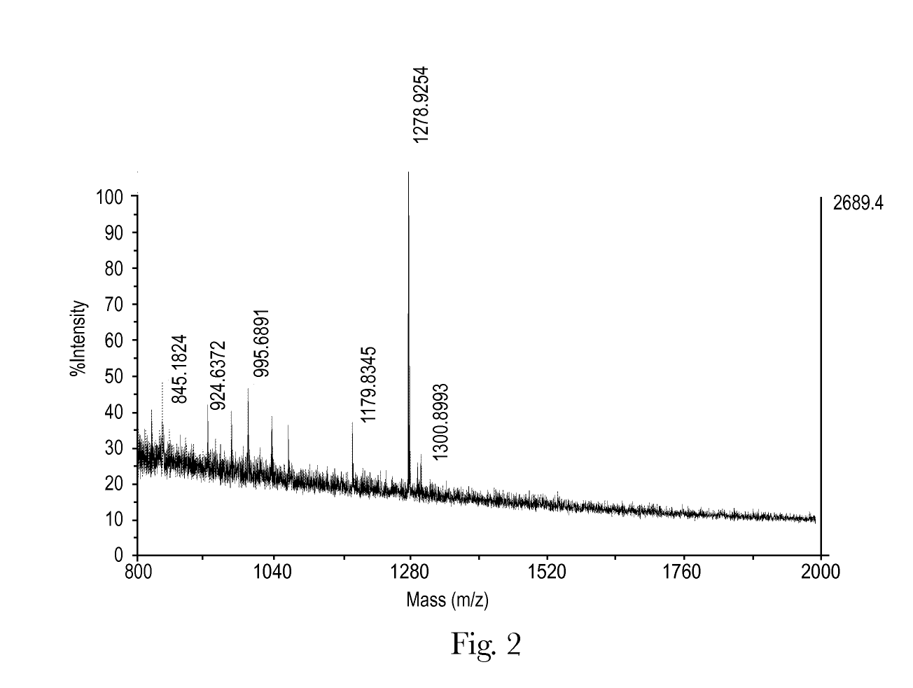

The soluble protein fraction of water extracts from chemically virgin, UV-exposed and bleach treated hair are examined by MALDI-TOF mass spectrometry to identify marker ions specific to UV or copper damaged hair. One marker in particular at m/z 1278 (FIG. 2) is found to occur in every UV-damaged sample (n=20). A small, but not appreciable, amount of this marker is also seen in the virgin and bleached hair. This is likely due to the natural weathering and UV exposure from the donor hair prior to being harvested. The fact that this marker ion intensity is increased with a controlled UV exposure indicates that it is a marker of UV-induced protein degradation and is likely due to oxidation of hair protein due to exposure to solar radiation.

Additionally, this method can also be used to indicate whether an individual's hair has a normal response to treatments. For example if an individual has a particular hair disease, a hair sample from this individual may not generate the same marker protein fragment as would a person who has a “normal” response. A person with a hair disease may generate additional, less and/or even different marker protein fragments than would be indicated by a normal response. A library of hair disease responses could also be created similar to that of the marker protein fragments for damage as described above. Therefore, a test for this hair disease could include exposing an individual's hair to a particular damaging treatment and then identifying the hair disease by the marker protein fragments that are generated from the damaging treatment.

In one embodiment of this hair protein loss test method the soluble and insoluble protein fragments are analyzed separately. Analyzing the soluble and insoluble protein fragments separately can result in higher sensitivity of protein fragment detection. Additionally, analyzing these protein fragments separately may further refine the determination of the location of the damage to the hair. To measure the soluble and insoluble protein fragments separately, after removal of the hair fibers the sample the in water can be centrifuged or the insoluble portion can be left to settle out from the soluble portion.

### Methods

### Hair Samples are Treated and Analyzed Using the Following Protocols:

Treatment of Hair with Copper: Chemically virgin hair is held under running water with nine-grain water hardness for 45 min. Three populations of hair tresses is created using water containing 0.00, 0.05 and 0.10 ppm copper, respectively. Prior to experiments, copper levels in hair are confirmed by inductively coupled plasma atomic spectroscopy (ICP-OES) measurements.

UV Treatment of Hair Tresses: UV exposure of copper treated hair is performed by irradiation with an Atlas Ci3000+weather-o-meter (Atlas, Chicago, Ill., US). An internal and outer quartz filter is used to simulate broad-spectrum, outdoor daylight with a specific irradiance of 1.48 W m2 at 420 nm. During the irradiation process, temperature and relative humidity is kept constant at 35° C. and 80% RH. Tresses are exposed for time indicated.

Sample Generation for MALDI-TOF: 0.2-0.3 g hair samples (2 in length) are collected from chemically virgin, UV and/or copper treated hair tresses and is added to glass scintillation vials. Distilled water is added at a ratio of 10:1 (mL water to g hair). Samples are shaken for 1 h at 2500 rpm on a DVX-2500 Multi-tube Vortexer platform (VWR International, Radnor, Pa., U.S.A.). Matrix-assisted laser desorption ionization time-of-flight (MALDI-TOF) mass spectrometry is used to detect peptide marker ions from the water extracts. Water extracts from the hair are mixed 1:1 with MALDI matrix α-cyano-4-hydroxycinnamic acid (α-cyano-4-hydroxycinnamic acid at 5 mg/mL in 80% acetonitrile/water/0.1% trifluroacetic acid). One microliter of this mixture is spotted on a target plate and allowed to air dry at room temperature before MALDI analysis. A MALDI-TOF/TOF 4800 Plus Mass Analyzer (AB-Sciex, Framingham, Mass., U.S.A.) is used in the positive ion reflectron mode. The mass spectrometer uses a 200-Hz frequency Nd:YAG laser, operating at a wavelength of 355 nm. Ions generated by the MALDI process are accelerated at 20 kV. MALDI-TOF mass spectra are generated in the mass range 800-4000 Da. Laser intensity is set between 3500 and 4000 V. Data is collected in an automated fashion using random sampling over the sample spot with 50 shots per subspectrum and a total of 1000 shots per spectrum. The intensity of peptide marker peaks for each extract is measured for quantitation.

Shampoo Formulation & Treatment: A simple surfactant shampoo is formulated containing 10.5% SLE1S, 1.5% SLS and 1.0% cocamidopropyl betaine surfactants. N,N′ethylenediamine disuccinnic acid (EDDS) at 0.1% active level is added to the test shampoo product as indicated. Color treated hair is washed for 20 cycles with a shampoo and then analysed for copper uptake using the ICP method. Each wash cycle consists of applying 0.1 g shampoo per gram of hair to the hair switch and lathering for 30 s followed by a 30 s rinse repeated for a total of two shampoo applications. Hair is then dried in a hot box at 80° C.

Effect of Copper Chelation on m/z 1278 Peptide Marker: Chemically virgin hair tresses are pre-dosed with copper for 45 minutes in water containing 0.05 ppm copper. Copper levels in hair is confirmed by ICP-OES measurements. Hair is washed for 20 cycles with shampoo with and without chelant (0.1% EDDS) and is exposed to artificial radiation for 3 hours after every 5 cycles. Each wash cycle consisted of applying 0.1 g/g shampoo to hair, lathering for 30 seconds, followed by a 30 second rinse, then repeated for a total of two shampoo applications. Hair is dried in a hot box at 80° C. between cycles.

Total Protein Loss Measurement: 0.2-0.3 g hair samples (2 in length) are collected from each hair tress and are added to glass scintillation vials. Distilled water is added at a ratio of 10:1 (mL water to g hair). Samples are shaken for 1 h at 2500 rpm on a DVX-2500 Multi-tube Vortexer platform (VWR International, Radnor, Pa., U.S.A.). Protein concentration in the extract is determined using the modified Lowry assay against a porcine gelatin standard (Modified Lowry Protein Assay kit supplied by Pierce, Rockford, Ill., U.S.A. http://www.piercenet.com).

Protein Fragment Identification: To identify and sequence the marker peptide m/z 1278 initially detected in MALDI-MS, a UV exposed hair water extract is analyzed using online NanoLC-High Resolution Orbitrap Mass Spectrometry, with a 60 min gradient, a 75 um i.d.×15 cm C18 reversed phase column and Easyspray ionization interface. The Orbitrap system provides a mass measurement accuracy better than 10 ppm. Mascot software is used to search Swiss-Prot database to identify the parent proteins of the marker fragment m/z 1278.7. Protein database search of the NanoLC-Orbitrap data is done with some common modifications, e.g., N-terminal acetylation, methione oxidation, amidation, etc.

### Results:

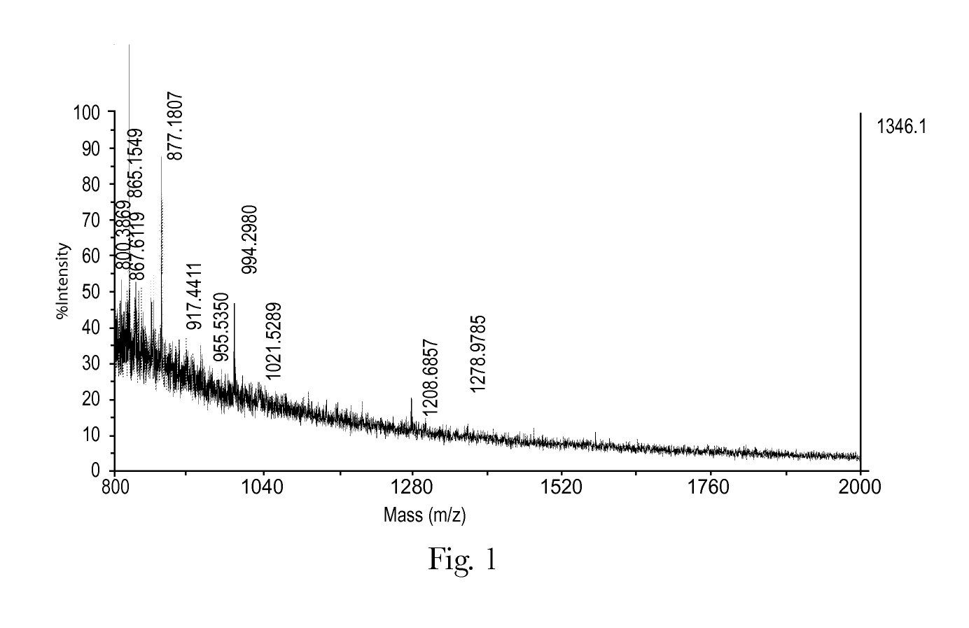

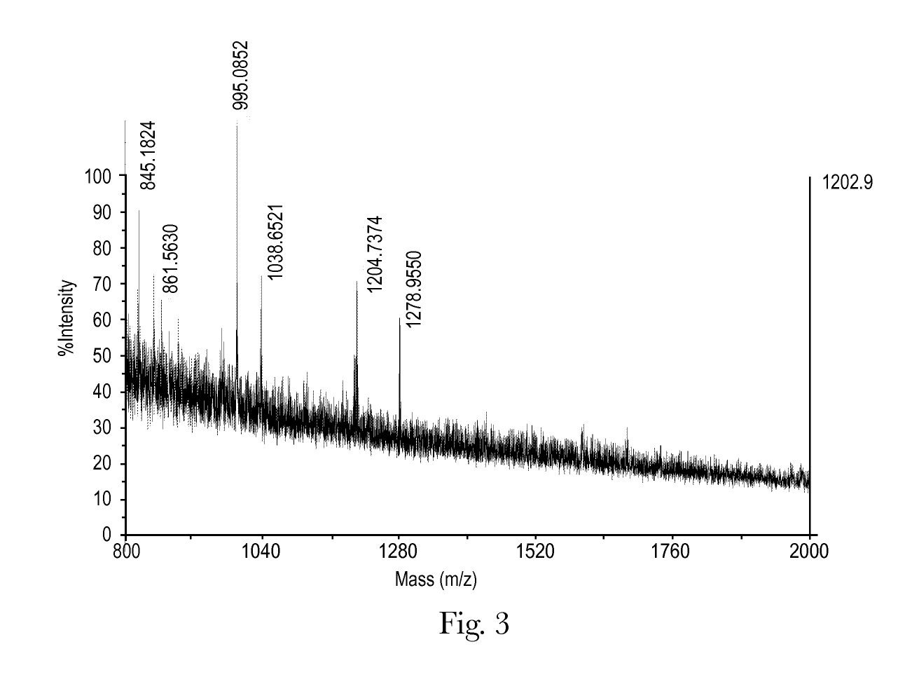

FIGS. 1, 2 & 3 demonstrate the MALDI-TOF spectra of extracts from virgin and treated hair samples. In FIG. 1 specific sets of fragments are released from Virgin Hair. As indicated in FIG. 2, specific sets of fragments are released for UV treated hair. And as indicated in FIG. 3, specific sets of fragments are released for bleached hair. Together these demonstrate that the m/z 1278 marker protein fragment is increased after UV treatment of the hair.

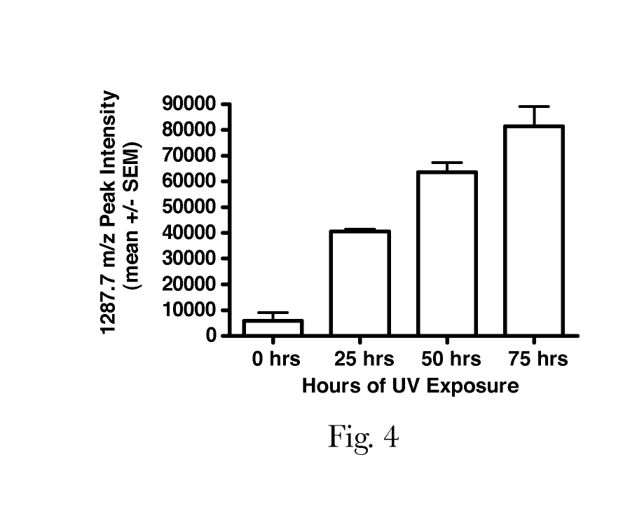

FIG. 4 demonstrates quantified MALDI-TOF signal intensity of the m/z 1278 biomarker from UV treated hair demonstrating the UV dose dependent generation of the m/z 1278 peak. The soluble protein fraction of water extracts from hair subjected to a time course of UV-exposure is examined by MALDI-TOF mass spectrometry. This data confirms that the m/z 1278 marker protein fragment increases in concentration as a function of UV exposure to virgin hair.

Table 1 demonstrates that the m/z 1278 marker protein fragment is also found to increase as a function of the length of UV exposure.

TABLE 1

| | Treatment | m/z 1278 intensity (mean of 2 |
|---|---|---|
| Hair Type | Time | samples) |
| Brown | No Treatment | 157 |
| Brown | 5 h | 819 |
| Brown | 10 h | 1383 |
| Brown | 20 h | 2347 |
| Brown | 30 h | 2597 |
| Brown | 40 h | 4906 |

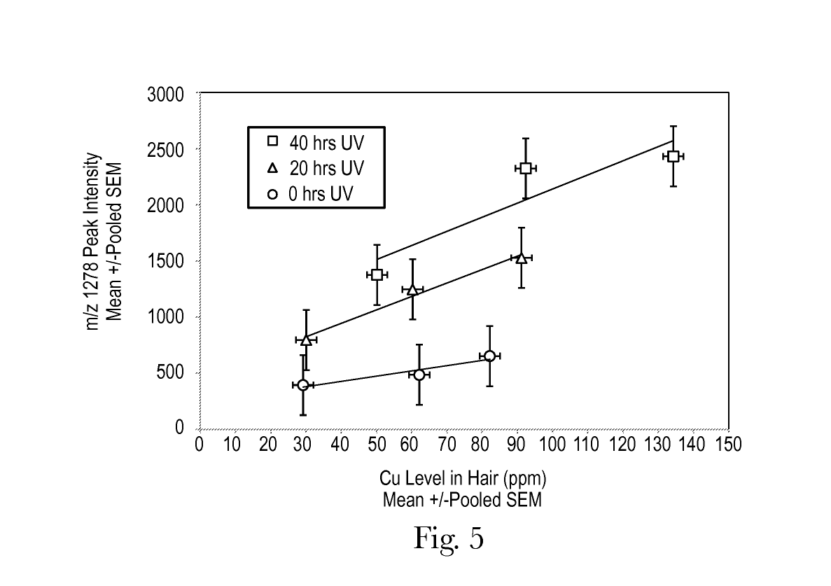

FIG. 5 demonstrates quantified MALDI-MS signal intensity of the m/z 1278 biomarker from copper-dosed, UV treated hair demonstrating the copper and UV dose dependent generation of the m/z 1278 peak. The dose response experiments are repeated with chemically untreated virgin hair dosed to different levels of copper to determine if the presence of redox metals in hair will increase the generation of the m/z 1278 marker peptide with UV exposure. The data show a clear positive correlation between copper level in virgin hair and presence of the m/z 1278 marker peptide after UV exposure. The correlation exists even in the absence of UV exposure (0 h), indicating that copper is playing a role in a non-UV-induced damage mechanism. These findings support a mechanism whereby copper serves as a catalyst for UV-induced oxidative breakdown of a specific hair protein by consistent liberation of the m/z 1278 peptide fragment. Final copper levels in hair are confirmed by inductively coupled plasma atomic spectroscopy (ICP-OES) measurements.

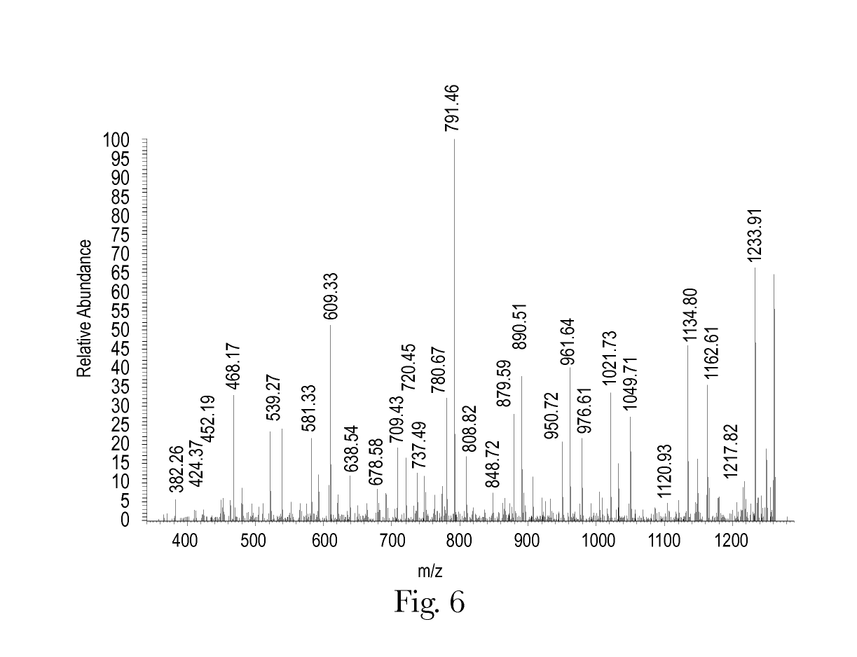

FIG. 6 demonstrates the MS/MS peptide sequencing of the m/z1278 biomarker using NanoLC-high resolution Orbitrap identifies S100A3 as the parent protein and indicating that this specific fragmentation of S100A3 is a unique biomarker for UV and/or copper stimulated hair damage. The UV damage marker ion m/z 1278 matches the sequence Ac-ARPLEQAVAAIV-NH2 (Molecular Weight=1277.7457), with 0.1 ppm accuracy to the calculated value (1277.7455), therefore an accurate sequence determination. The mass spectrometric fragment ions from the marker m/z 1278.7 shows mainly a series ions, e.g., 452.2, 581.3, 709.4, 780.7, etc and b series ions, e.g., 609.3, 737. 4, 1049.7, 1162.8, etc, due to the arginine residue located near the N-terminal of this peptide. In addition to the singly charged ion m/z 1278.7, the doubly charged ion m/z 640.3 of this marker peptide is also detected in the same nanoLC-Orbitrap analysis, with a mass measurement accuracy of −0.07 ppm. Mascot software is used to search Swiss-Prot protein database to identify that the parent protein of the marker fragment m/z 1278 is S100A3. S100A3 has been identified in the literature as an integral hair cuticle protein and important to that integrity of the hair shaft. This data demonstrates the specific nature of the S100A3 fragmentation by generation of the m/z 1278 marker peptide and its relationship to intra-hair copper concentration. Subsequent structural analysis of the published S100A3 crystal structure shows that the location of the metal ion binding site (i.e. copper and zinc) in the dimer form of the protein is located in close proximity to the m/z 1278 fragment cleavage site, indicating that the proximity of the copper to the peptide fragmentation is a factor in the specific fragmentation of S100A3 seen in this work.

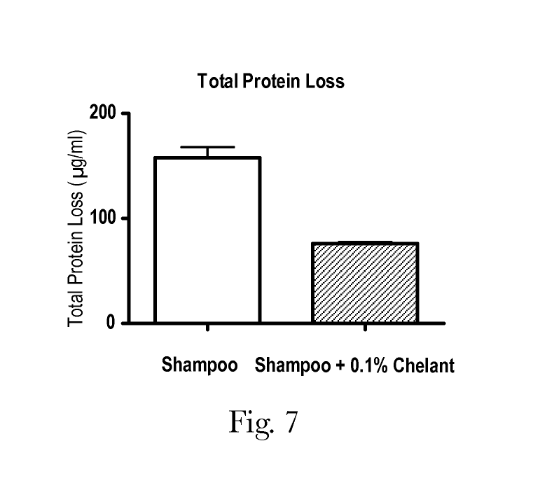

FIG. 7 demonstrates UV stimulated protein loss decreases in hair tresses with pre-treatment of a shampoo that contains a copper chelant. Chelants are well known for controlling metal-induced radical formation by preventing the 1-electron catalytic cycling of the redox metal between its two accessible oxidation states. Previous work has shown that EDDS (N,N′-ethylenediamine disuccinic acid) can prevent copper-induced radical formation in the oxidation conditions used during hair colouring. FIG. 7 demonstrates that a copper loaded hair washed in a shampoo containing 0.1% EDDS can reduce the levels of UV-initiated total protein loss by preventing the accumulation of catalytic copper ions.

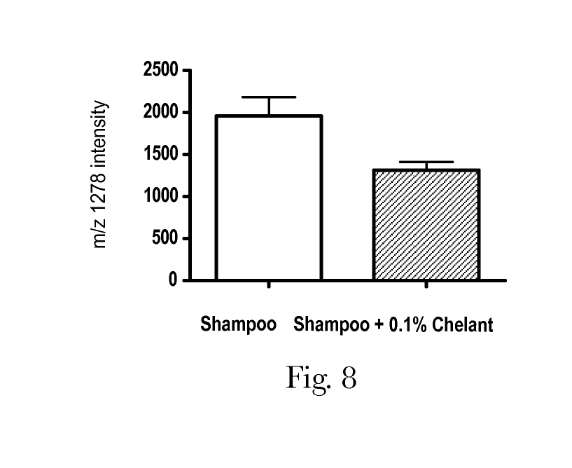

FIG. 8 demonstrates UV stimulated m/z 1278 biomarker generation decreases in hair tresses with pre-treatment of a shampoo that contains a copper chelant. Utilizing the same water hair extracts as in FIG. 7, the MALDI-MS spectrum shows that EDDS (N,N′-ethylenediamine disuccinic acid) at 0.1% in a shampoo can reduce copper-induced formation of the m/z 1278 peptide marker of UV-initiates hair damage, again demonstrating the specificity of fragmentation and relative sensitivity of the m/z 1278 peptide marker for copper levels inside the hair.

In an embodiment of the present invention, various treatments may be used to reduce UV damage to hair. Non-limiting examples of such treatments are:

Chelating Agents

In an embodiment of the present invention, the treatment composition may comprise a chelant. The term “chelant” (or “chelating agent” or “sequestering agent”) is well known in the art and refers to a molecule or a mixture of different molecules each capable of forming a chelate with a metal ion. A chelate is an inorganic complex in which a compound (chelant) is coordinated to a metal ion at two or more points so that there is a ring of atoms including the metals. Chelants contain two or more electron donor atoms that form the coordination bonds with the metal ion.

Any chelants is suitable for use herein. Chelants are well known in the art and a non-exhaustive list thereof can be found in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996) both incorporated herein by reference and disclosed in U.S. Pat. No. 5,747,440 Kellett et al.

Examples of aminocarboxylic acid chelants suitable for use herein include nitrilotriacetic acid and polyaminocarboxylic acids such as diethylenetriamine pentaacetic acid (DTPA), ethylenediamine disuccinic acid (EDDS), ethylenediamine diglutaric acid (EDGA), 2-hydroxypropylenediamine disuccinic acid (HPDS), glycinamide-N,N′-disuccinic acid (GADS), ethylenediamine-N-N′-diglutaric acid (EDDG), 2-hydroxypropylenediamine-N-N′-disuccinic acid (HPDDS), ethylenediaminetetraacetic acid (EDTA), salts thereof and derivatives thereof. Other suitable aminocarboxylic type chelants for use herein are iminodiacetic acid derivatives such as N-2-hydroxyethyl N,N diacetic acid or glyceryl imino diacetic acid (described in EP-A-317,542 and EP-A-399,133), iminodiacetic acid-N-2-hydroxypropyl sulfonic acid and aspartic acid N-carboxymethyl N-2-hydroxypropyl-3-sulfonic acid (described in EP-A-516,102), β-alanine-N,N′-diacetic acid, aspartic acid-N,N′-diacetic acid, aspartic acid-N-monoacetic acid and iminodisuccinic acid chelants (described in EP-A-509,382), ethanoldiglycine acid, salts thereof and derivatives thereof.

EP-A-476,257 describes suitable amino based chelants. EP-A-510,331 describes suitable chelants derived from collagen, keratin or casein. Further non-limiting examples include a suitable alkyl iminodiacetic acid chelants. Dipicolinic acid and 2-phosphonobutane-1,2,4-tricarboxylic acid are also suitable.

Preferred amino carboxylic chelants include diamine-N,N′-dipolyacid and monoamine monoamide-N,N′-dipolyacid chelants, salts thereof and derivatives thereof. Preferred polyacids contain at least two acid groups independently selected from the carboxyl group (—COOH), the o-hydroxyphenyl group, the m-hydroxyphenyl group and the p-hydroxyphenyl group. Preferably at least one acid moiety is a carboxylic moiety. Suitable polyacids include diacids, triacids and tetraacids, preferably diacids. Preferred salts include alkali metal, alkaline earth, ammonium or substituted ammonium salts. EDTA is a tetramonoacid and does not belong to this class of preferred chelants.

Preferred aminocarboxylic acid for use herein are diamine-N,N′-dipolyacids and monoamine monoamide-N,N′-dipolyacids wherein the polyacid species is a diacid, preferably a diacid having a carbon chain length of from about 3 to about 10 carbon atoms, more preferably from about 4 to about 6 carbon atoms, even more preferably about 4 carbon atoms. Exemplary diamine dipolyacids suitable for use herein include ethylenediamine-N,N′-disuccinic acid (EDDS), ethylenediamine-N,N′-diglutaric acid (EDDG), 2-hydroxypropylenediamine-N,N′-disuccinic acid (HPDDS), all disclosed in European Patent EP0687292, and EDDHA (ethylenediamine-N-N′-bis(ortho-hydroxyphenyl acetic acid), a method of preparation of which is disclosed in EP331556. A preferred monoamine monoamide-N,N′-dipolyacid is glycinamide-N,N′-disuccinic acid (GADS), described in U.S. Pat. No. 4,983,315.

Highly preferred for use herein is ethylenediamine-N,N′-disuccinic acid (EDDS), derivatives and salts thereof. Preferred EDDS compounds for use herein are the free acid form, and salts thereof. Preferred salts include alkali metal, alkaline earth metals, ammonium or substituted ammonium salts. Highly preferred salts are sodium, potassium, magnesium and calcium salts. Examples of such preferred sodium salts of EDDS include Na₂EDDS and Na₃EDDS.

Highly preferred for use herein is ethylenediamine-N,N′-disuccinic acid (EDDS), derivatives and salts thereof. Preferred EDDS compounds for use herein are the free acid form, and salts thereof. Preferred salts include alkali metal, alkaline earth metals, ammonium or substituted ammonium salts. Highly preferred salts are sodium, potassium, magnesium and calcium salts. Examples of such preferred sodium salts of EDDS include Na₂EDDS and Na₃EDDS.

The structure of the acid form of EDDS is as follows:

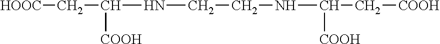 [SMILES: O=C(O)CC(NCCNC(CC(=O)O)C(=O)O)C(=O)O]

EDDS can be synthesised, for example, from readily available, inexpensive starting materials such as maleic anhydride and ethylenediamine. The synthesis of EDDS from maleic anhydride and ethylene diamine yields a mixture of three optical isomers, [R,R], [S,S], and [S,R] (25% S,S, 50% R,S and 25% R,R), due to the two asymmetric carbon atoms. The biodegradation of EDDS is optical isomer-specific, with the [S,S] isomer degrading most rapidly and extensively. Preferred chelants that are not diamine-N,N′-dipolyacid and monoamine monoamide-N,N′-dipolyacid chelants include N,N′-bis(2-hydroxybenzyl)ethylenediamine-N,N′-diacetic acid (HBED) salts thereof and derivatives thereof:

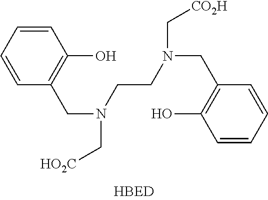 [SMILES: O=C(O)CN(CCN(CC(=O)O)Cc1ccccc1O)Cc1ccccc1O]

Examples of suitable HBED derivatives can be found in WO9744313, assigned to Novartis.

Suitable polyphosphoric acid chelants include molecules which contain more than one P atom and have P—O—P bonds. Polyphosphoric acid chelants and salts (polyphosphates) can be linear and are generally represented by the formula [PₙO₃ₙ₊₁]⁽ⁿ⁺²⁾⁻M₍ₙ₊₂₎⁺ wherein M is a suitable counter-ion such as H⁺, Na⁺ or K⁺ and n an integer. Polyphosphoric acid type chelants and their polyphosphate salts can also be cyclic and have the formula [PₙO₃ₙ]ⁿ⁻Mₙ⁺. Representative examples include, among other, sodium tripolyphosphate, tetrasodium diphosphates, hexametaphosphoric acid and sodium metaphosphate.

Suitable phosphonic acid chelants include amino alkylene poly (alkylene phosphonic acid), ethane 1-hydroxy diphosphonic acids and nitrilo trimethylene phosphonic acids, salts thereof and derivatives thereof. Suitable chelants of this type are disclosed in U.S. Pat. No. 4,138,478, Reese et al., U.S. Pat. No. 3,202,579 and U.S. Pat. No. 3,542,918, Berth et al, all incorporated herein by reference.

Preferred phosphonic acid type chelants for use herein have the formula (I) below:

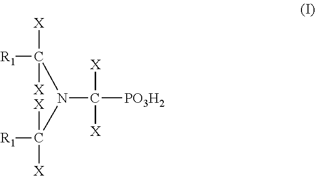

wherein each X are independently selected from hydrogen or alkyl radicals, preferably hydrogen or alkyl radicals having from 1 to 4 carbon atoms, preferably hydrogen; and each R₁ are independently selected from —PO₃H₂ or a group having the formula (II) below:

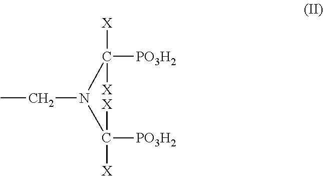

Preferred chelants according to Formula (I) for use herein are aminotri-(1-ethylphosphonic acid), ethylenediaminetetra-(1-ethylphosphonic acid), aminotri-(1-propylphosphonic acid), aminotri-(isopropylphosphonic acid) and chelants having the formula (III) below:

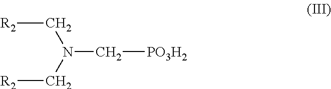

wherein each R₂ are independently selected from —PO₃H₂ or a group having the formula (IV) below:

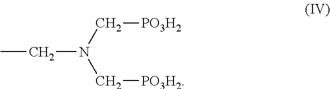

Especially preferred chelants according to formula (IV) for use herein are aminotri-(methylenephosphonic acid), ethylene-diamine-tetra-(methylenephosphonic acid) (EDTMP) and diethylene-triamine-penta-(methylenephosphonic acid) (DTPMP).

Other chelants suitable for use are amino acids such as histidine, arginine, lysine, asparagine, tryptophan, serine, glutamine, alanine, glycine and proline. A further non-limiting example is zinc pyrithione or metal salts of pyrithione.

Levels of the chelant in the treatment compositions can be as low as about 0.01 wt % or even as high as about 10 wt %, but above the higher level (i.e., 10 wt %) formulation and/or human safety concerns may arise. In an embodiment, the level of the chelant may be at least about 0.05 wt %, at least about 0.1 wt %, at least about 0.25 wt %, at least about 0.5 wt %, at least about 1 wt %, or at least about 2 wt % by weight of the treatment composition. Levels above about 4 wt % can be used but may not result in additional benefit.

### Sunscreens

In an embodiment of the present invention, a treatment composition may include suitable sunscreens which includes, but are not limited to: aminobenzoic acid, avobenzone, cinoxate, dioxybenzone, homosalate, menthyl anthranilate, octocrylene. octyl methoxycinnamate, octyl salicylate, oxybenzone, padimate O, phenylbenzimidazole sulfonic acid, sulisobenzone, titanium dioxide, trolamine salicylate, zinc oxide and combinations thereof.

Typical levels of sunscreens in hair care products may range from about 0.1% to about 25% by weight of a hair care composition.

### Divalent Salts

In an embodiment of the present invention, a treatment may include suitable examples of divalent salts which includes, but are not limited to divalent salts of magnesium, zinc, or calcium such as those having a gluconate, chloride, citrate, carbonate, or acetate counterion. The salt may be in anhydrous or hydrated form.

Suitable levels of divalent salts may be in the range of about 0.01 wt % or even as high as about 10 wt %.

### Anti-Oxidants/Radical Scavengers

The compositions of the present invention may include a safe and effective amount of an anti-oxidant/radical scavenger. The anti-oxidant/radical scavenger is especially useful for providing protection against UV radiation that can cause increased scaling or texture changes in the stratum corneum and against other environmental agents, which can cause skin damage. A safe and effective amount of an anti-oxidant/radical scavenger may be added to the compositions of the subject invention, preferably from about 0.1 wt % to about 10 wt %, more preferably from about 1 wt % to about 5 wt %, of the composition.

Anti-oxidants/radical scavengers such as ascorbic acid (vitamin C) and its salts, ascorbyl esters of fatty acids, ascorbic acid derivatives (e.g., magnesium ascorbyl phosphate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename TroloxR), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, lysine, methionine, proline, superoxide dismutase, silymarin, tea extracts, grape skin/seed extracts, melanin, and rosemary extracts may be used. Preferred anti-oxidants/radical scavengers are selected from tocopherol sorbate and other esters of tocopherol, more preferably tocopherol sorbate. For example, the use of tocopherol sorbate in topical compositions and applicable to the present invention is described in U.S. Pat. No. 4,847,071.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method to measure ultraviolet damage of hair comprising:
a) eluting a protein fragment from a hair sample with an aqueous solution;
b) extracting the proteins using a solvent;
c) analyzing the protein fragment samples with MALDI-MS; resulting in protein fragment results;
d) identifying presence of a marker protein fragment and identifying what the fragment is by indentifying the amino acid sequence using high resolution Orbitrap-MS wherein the protein fragment is a protein fragment of S100A3.

2. A method according to claim 1 wherein the protein fragment of S100A3 is has a m/z 1278.

3. A method according to claim 1 wherein the protein fragment of S1000A3 has an amino acid sequence as acetylated-ARPLEQAVAAIV-Amide.

4. A method to identify a treatment for ultraviolet damage of hair comprising
a) applying a treatment composition to hair sample A; apply no treatment composition to a sample B;
b) applying ultraviolet light to a hair sample A and hair sample B;
c) eluting a protein fragment from hair samples with an aqueous solution;
d) extracting the proteins using a solvent;
e) identifying or measuring the marker protein fragments by identifying the unique modification patterns which exist in sample wherein the MALDI-MS protein fragment results in a protein fragment of S100A3 wherein Sample A has a decrease in protein fragment of S100A3 compared to Sample B.

5. The method of claim 4 wherein the treatment composition comprises a chelator.

6. The method of claim 5 wherein the chelator is selected from the group consisting of ethylenediamine-N,N′-disuccinic acid (EDDS), ethylenediamine-N,N′-diglutaric acid (EDDG), 2-hydroxypropylenediamine-N,N′-disuccinic acid (HPDDS), glycinamide-N,N′-disuccinic acid (GADS), ethylenediamine-N-N′-bis(ortho-hydroxyphenyl acetic acid (EDDHA), histidine, arginine, lysine, asparagine, tryptophan, serine, glutamine, alanine, glycine and proline, zinc pyrithione or metal salts of pyrithione, salts thereof, derivatives thereof, and mixtures thereof.

7. The method of claim 4 wherein the treatment composition comprises an anti-oxidant.

8. The method of claim 7 wherein the anti-oxidant is selected from the group consisting of ascorbic acid and its salts, ascorbyl esters of fatty acids, ascorbic acid derivatives, tocopherol, tocopherol sorbate, tocopherol acetate, other esters of tocopherol, butylated hydroxy benzoic acids and their salts, 6-hydroxy-2,5,7,8-tetramethylchroman-2-carboxylic, gallic acid and its alkyl esters, uric acid and its salts and alkyl esters, sorbic acid and its salts, lipoic acid, amines, sulfhydryl compounds, dihydroxy fumaric acid and its salts, lycine pidolate, arginine pilolate, nordihydroguaiaretic acid, bioflavonoids, lysine, methionine, proline, superoxide dismutase, silymarin, tea extracts, grape skin/seed extracts, melanin, rosemary extracts and mixtures thereof.

9. The method of claim 4 wherein the treatment composition comprises a sunscreen.

10. The method of claim 9 wherein the sunscreen is selected from the group consisting of aminobenzoic acid, avobenzone, cinoxate, dioxybenzone, homosalate, menthyl anthranilate, octocrylene. octyl methoxycinnamate, octyl salicylate, oxybenzone, padimate O, phenylbenzimidazole sulfonic acid, sulisobenzone, titanium dioxide, trolamine salicylate, zinc oxide and mixtures thereof.

11. The method of claim 4 wherein the treatment composition comprises a divalent metal selected from the group consisting of magnesium, zinc, calcium and salts thereof and mixtures thereof.

12. A method to measure copper damage of hair comprising:
a) eluting a protein fragment from a hair sample with an aqueous solution;
b) extracting the proteins using a solvent;
c) analyzing the protein fragment samples with MALDI-MS; resulting in protein fragment results;
d) identifying presence of a marker protein fragment and identifying what the fragment is by indentifying the amino acid sequence using high resolution Orbitrap-MS wherein the protein fragment is a protein fragment of S100A3.

13. A method to measure ultraviolet damage of hair comprising:
analyzing hair with MALDI-Imaging; resulting in protein fragment results.

14. A method to measure copper damage of hair comprising:
analyzing hair with MALDI-Imaging; resulting in protein fragment results.

15. A method according to claim 1 wherein the method is used to support marketing or advertising claims.
